(19) United States
(12) Patent Application Publication
(10) Pub. No.: US 2005/0277836 A1
(43) Pub. Date: Dec. 15, 2005

(54) Transesophageal ultrasound transducer probe

(76) Inventors: Timothy L. Proulx, Santa Cruz, CA (US); Lewis J. Thomas III, Palo Alto, CA (US); Walter T. Wilser, Cupertino, CA (US); Mirsaid Bolorforosh, Schenectedy, NY (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/051,089

(22) Filed: Feb. 4, 2005

Related U.S. Application Data
(60) Provisional application No. 60/542,388, filed on Feb. 5, 2004.

(51) Int. Cl. A61B 8/14

(52) U.S. Cl. 600/466

(57) ABSTRACT

A multi-dimensional, such as a two-dimensional, array is provided within a transesophageal probe housing. Since the size of the transesophageal probe housing is limited for comfort of a patient, active electronics are positioned spaced away from the multi-dimensional transducer array, such as within a handle of the transesophageal probe housing. Signal conductors connect the multi-dimensional transducer array to the active electronics. The elements of the multi-dimensional transducer array have a capacitance much lower than parasitic capacitance of the connecting cables. To provide a higher signal-to-noise ratio, the elements are formed from multiple layers of transducer material, increasing the capacitance of each element.

### REFERENCE TO RELATED APPLICATIONS

The present patent document claims the benefit of the filing date under 35 U.S.C. § 119(e) of Provisional U.S. Patent Application Ser. No. 60/542,388, filed Feb. 5, 2004, which is hereby incorporated by reference.

### BACKGROUND

The present invention relates to transesophageal probes. In particular, ultrasound transducer probes for imaging from within a patient are provided.

Transesophageal probes are designed for insertion in the esophagus of a person. For ultrasound imaging, a transducer array is positioned on a distal end of the transesophageal probe. Once the probe is inserted within the esophagus of a patient, the transducer array is positioned adjacent to the esophagus. The heart or other internal organs may be imaged.

The transducer array on transesophageal probes may have one or two linear arrays, such as two linear arrays in a cross pattern. Alternatively or additionally, a mechanical turn table is provided for moving a linear array to different positions. Different images of the heart may be generated using two different positions of the linear array.

### BRIEF SUMMARY

By way of introduction, the preferred embodiments described below include ultrasound transducers for imaging within an esophagus and methods for ultrasound imaging from within a patient. A multi-dimensional, such as a two-dimensional, array is provided within a transesophageal probe housing. Since the size of the transesophageal probe housing is limited for comfort of a patient, active electronics are positioned spaced away from the multi-dimensional transducer array, such as within a handle of the transesophageal probe housing. Signal conductors connect the multi-dimensional transducer array to the active electronics. The elements of the multi-dimensional transducer array have a capacitance much lower than parasitic capacitance of the connecting cables. To provide a higher signal-to-noise ratio, the elements are formed from multiple layers of transducer material, increasing the capacitance of each element. Spacing electronics in a portion of a transesophageal probe maintained outside of a patient and using multi-layer transducer elements in a multi-dimensional transducer array may be used independently of each other in different embodiments. Alternatively, both features are used in a same embodiment.

In a first aspect, an ultrasound transducer is provided for imaging within an esophagus. A multi-dimensional transducer array is in or on a transesophageal probe housing. The multi-dimensional transducer array has a plurality of elements. At least one of the elements has multiple layers of transducer material.

In a second aspect, an ultrasound transducer is provided for imaging within an esophagus. A transesophageal probe housing has a distal end, a handle and a middle section between the handle and distal end. The distal end and the middle section are free of active electronics. A multi-dimensional transducer array at or adjacent to the distal end has a plurality of elements. Active electronics in the handle electrically connect with the multi-dimensional transducer array.

In a third aspect, a method is provided for ultrasound imaging from within a patient. An electrical impedance of a plurality of signal conductors is substantially-matched with a respective plurality of elements of a multi-dimensional transducer array in a transesophageal ultrasound probe. The matching is provided by each of the plurality of elements having at least two layers of transducer material. Active electronics are positioned in a portion of the transesophageal ultrasound probe maintained outside the patient. The signal conductors electrically connect the active electronics with the elements.

The present invention is defined by the following claims, and nothing in this section should be taken as a limitation on those claims. Further features, aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a perspective view of one embodiment of a transesophageal probe; and

FIG. 2 is a perspective view of a multi-dimensional transducer array with multi-layer elements.

### DETAILED DESCRIPTION OF THE DRAWINGS AND PRESENTLY PREFERRED EMBODIMENTS

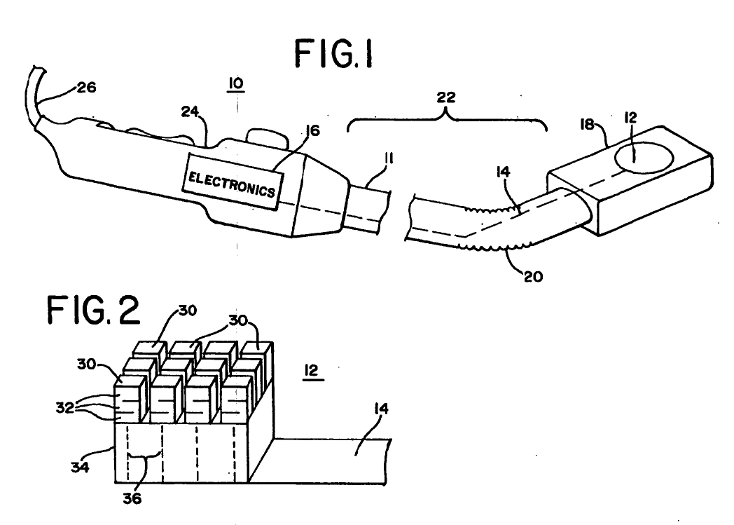

FIG. 1 shows one embodiment of a transesophageal ultrasound probe 10. The transesophageal ultrasound probe 10 is an ultrasound transducer for imaging within an esophagus of a patient. The probe 10 includes a transesophageal probe housing 11, a multi-dimensional transducer array 12, signal conductors 14 and active electronics 16. Additional, different or fewer components may be provided. The probe 10 is sized and shaped for insertion through the mouth into the esophagus of a patient. Coaxial cables in a cable 26 electrically connect the probe 10 to an ultrasound imaging system. The ultrasound imaging system receives signals from the cable 26 for generating two-dimensional images or three-dimensional representations. Since a multi-dimensional transducer array 12 is provided, the imaging system may generate three-dimensional representations in real time or as data is acquired. The imaging system generates ten or more, such as 25 or more, frames or images as second to provide real time three-dimensional imaging.

The transesophageal probe housing 11 is Pebax®, plastic, silicon, metal, stainless steel, epoxy, fiberglass, combinations thereof or other now known or later developed material. The housing 11 includes a distal end 18, a middle section 22 within an articulating section 20, a handle 24 and a connector for connecting the cable 26 to the imaging system. Each of these sections is of a same or different material or combinations of materials. For example, the distal end 18 and the middle section 22 are any now known or later developed material operable to easily slide within the esophagus while minimizing discomfort or risk of damage to a patient. The middle section 22 may be generally rigid, completely rigid, or flexible. The middle section 22 is hard or soft, such as associated with a thin covering. The handle 24 is plastic, rubber, foam or combinations thereof for ease of handling and manipulation by a user. The distal end 18 includes a generally flat surface or other structure for allowing positioning of the multi-dimensional transducer array 12 adjacent to tissue within the patient.

The middle section is 40 or more centimeters in length, such as being 80 centimeters to a full meter in length. In alternative embodiments, a lesser or greater length is provided. The middle section 22 is of a set length or may expand or contract to provide a variable length. The articulating section 20 is a more flexible section than the remainder of the middle section 22 and/or includes mechanical components, such as a joint, for allowing bending on a single or two axes orthogonal to the general length of the middle section 22. The articulating section 20 is steerable, such as connecting with one or more steering wires to a knob on the handle 24 for guiding the distal end 18. In one embodiment, the articulating section 20 is immediately adjacent to the distal end 18, but may be positioned elsewhere along the middle section 22.

The middle section 22 and distal end 18 are sized to allow positioning within the esophagus of a patient. For example, the maximum diameter anywhere along the length of the middle section 22 or the distal end 18 is 16 millimeters, 20 millimeters or a greater or lesser number. For example, the distal end 18 has a width of 13 millimeters, a height of 10 millimeters and a length of 32 millimeters. The middle section 22 has a similar or lesser diameter. Even smaller transesophageal diameter dimensions may be provided for greater patient comfort, such as a maximum of 10 or 12 millimeters.

The multi-dimensional transducer array 12 is an array of piezoelectric elements. Multi-dimensional includes an arrangement of M×M elements where both N and M are greater than 1. N and M may be equal or unequal values. Other two-dimensional arrays, such as disclosed in U.S. Pat. Nos. 6,503,204, 6,582,367, 6,679,849, 6,572,547, the disclosures of which are incorporated herein by reference, may be used. The elements are positioned on a flat planar surface or along a curved surface. Any number of elements may be provided, such as a 32 element by 32 elements two-dimensional array. Six hundred or more, 796, 768 or other numbers of elements may be provided. Fewer number of elements may be used, such as associated with a sparsely sampled array. An example sparse array is an array with a spiral distribution of elements, such as disclosed in U.S. Pat. No. 6,359,367, the disclosure of which is incorporated herein by reference. The elements are distributed in rectangular, triangular, hexagonal or other grid pattern. For hexagonal, triangular or other grid patterns, a plurality of elements is distributed along two different dimensions. The array may have a square, circular, oblong, rectangular or other outer periphery shape. The array 12 is positioned within the distal end 18 of the probe 10. Where the middle section 22 and a distal end 18 have a similar shape, the array 12 is positioned at or adjacent to the distal end of the probe.

The array 12 is operable at any desired ultrasound frequency, such as a frequency centered at 2 to 10 Megahertz. For example, the elements are 250 by 250 microns in an 8 millimeter by 8 millimeter two-dimensional array for 5 MHz operation. Other size elements or arrays with the same or different number of elements may be provided.

In one embodiment, each of the elements of the array 12 is a single layer of piezoelectric material. In alternative embodiments shown in FIG. 2, each of the elements 30 includes multiple layers 32 of transducer material, such as piezoelectric ceramics, piezoelectric single crystals or electrostrictors. Two or more layers, such as at least three or four layers 32 are provided for each element 30, increasing the capacitance of an element. Each of the elements 30 of the entire array 12 are multilayer elements, but one or more elements may have fewer number or a greater number of layers, such as having a single layer. As shown in FIG. 2, each of the elements 30 is operable in a k₃₃ or longitudinal extensional mode where the layers are stacked along a range or depth dimension of the array 12.

Such arrays may be manufactured using any of the techniques disclosed in U.S. Pat. Nos. 6,656,124; 5,548,564; 5,381,385; 5,834,880; or 5,704,105, the disclosures of which are incorporated herein by reference. Prefabricated multilayer posts are provided for each element. Each element is placed onto a conductive pad on the backing block to build the array. Alternatively, vias are formed within the arrays for electrically connecting multiple layers of electrodes. In yet another embodiment, multiple ceramic layers are tape casted. Vias are drilled and plated at sub-element dimensions to short electrodes on every other layer. The vias are then diced through in each direction after bonding. Other now known or later developed techniques may be used for forming the multilayer transducer array, such as picking and placing individual multilayer posts, drilling and plating vias, patterning multilayer electrodes with dicing and plating of kerfs, or using modular groups of elements positioned within a frame together to form the multi-dimensional transducer array 12. In an alternative embodiment, each of the elements 30 is operable in a k₃₁ resonant mode, such as providing layers 32 stacked along a horizontal dimension or perpendicular to a longitudinal displacement direction. For example, a multi-dimensional transducer array operable in a 31 resonant mode disclosed in U.S. Pat. No. ______ (application Ser. No. ______ (Attorney Reference No. 2005P00039US)) or U.S. Pat. No. 6,288,477, the disclosures of which are incorporated herein by reference, is used.

The array 10 includes the transducer material or elements 30 and additional layers within a transducer stack. For example, one or more matching layers with or without a window or lens are provided on a top of each of the elements 30. For example, two matching layers are provided. One is high impedance matching layer of conductive graphite and the other is a conductive or nonconductive low impedance matching layer formed over a ground foil. A flexible circuit for providing a ground plane, or a conductive matching layer is used for connecting with one of two sets of electrodes provided on each of the elements 30. A backing block 34 is positioned beneath the elements 34 for absorbing acoustic energy transmitted away from a patient. A flexible circuit or other structure is provided between the backing block and the elements 30 for connection with another of the sets of electrodes of each element 30. Alternatively and as shown in FIG. 2, the backing block 34 includes Z-axis connectors, such as conductors formed within the backing block for routing signals to the conductors 14. Z-axis conductive backing material with drilled vias, laminated conductor layers or some other now known or later developed technique provides conductive traces to each element 30. The conductors 14 are a flexible circuit, ribbons of conductors, coaxial cables or other now known or later developed structures. For example, a flexible circuit connects with a plurality of unshielded cables 14.

Referring to FIGS. 1 and 2, a plurality of signal conductors extend from the multi-dimensional transducer array 12 through the middle section 22 to the active electronics 16. The conductors 14 comprise coaxial cables, ribbons, flexible circuits, unshielded cables or other now known or later developed signal conductors. In one embodiment, electromagnetic inference between the various conductors is avoided by randomized wrapping or positioning of unshielded cables along the length of the middle section 22. A ground plane or shield may be provided around the group of unshielded cables for reducing interference from external sources. Alternatively, coaxial cables with a small gauge, such as a 52 or 54 gauge are provided. Ribbons with an associated ground plane formed therein may alternatively be used.

A separate signal conductor 14 is provided for each active element 30 of the multi-dimensional transducer array 12. For example, where the array 12 includes at least 600 elements, at least 600 signal conductors 14 are provided for connection with the elements 30 in the active electronic 16. Where some of the elements are not used, a fewer number of signal conductors may be connected with the array 12. Alternatively, the channel count or number of signal conductors 14 is reduced by providing a sparse sampling of the array 12 or using a smaller array 12. Transmit or receive signals for a given element are provided along the signal conductors 14.

The active electronics 16 are transistors, high voltage switches, amplifiers, mixers, delays, buffers, phase rotators, processors, waveform generators, controllers, combinations thereof or other now known or later developed active electronic device. The active electronics 16 are implemented in one or more application specific integrated circuits, processors, digital circuits, analog circuits, field programmable gate arrays, or combinations thereof. The active electronics 16 are positioned with the handle 24. Alternatively, the active electronics 16 are positioned within an image system separate from the probe 10. The distal end 18 and the middle section 22 are free of active electronics, such as providing a direct electrical connection from the array 12 to the active electronics 16 in the handle 24 without pre-amplification or other active electrical processes adjacent to the transducer array 12. The signal conductors 14 electrically connect the active electronics 16 to the multi-dimensional array 12.

The active electronics 16 perform one or more functions. For example, the active electronics 16 include pre-amplifiers for amplifying signals received on the conductors 14 from the elements 30 of the array 12. As another example, multiplexers are provided for switchably connecting different signal conductors 14 and associated elements 30 of the array 12 to different channels of the electronics 16 or coaxial cables within the cable 26. As another example, the active electronics 16 implement a transmit/receive switch. As yet another example, the active electronics 16 implement a portion or entire transmit beamformer, such as providing a plurality of transistors for generating transmit waveforms. Amplifier and/or buffer components may be used for relatively delaying or applying apodization across different channels of the transmit beamformer function. As yet another-example, some or all of the components of the receive-beamformer are implemented by the active electronics 16. Buffers, phase rotators and/or amplifiers are provided for relatively delaying and/or apodizing receive signals. One or more summers combine the signals from different elements 30 to form beamformed or sub-array beamformed information.

In one embodiment, the active electronics 16 are operable to combine signals from a plurality of elements onto a fewer number of outputs, such as associated with sub-array beamforming or time division multiplexing. For example, the active electronics 16 implement a plurality of mixers for mixing signals associated with a plurality of different sub-arrays, such as disclosed in U.S. Pat. No. (application Ser. No. 10/788,021) or U.S. Pat. No. 5,573,001, the disclosures of which are incorporated herein by reference. Any number of combinations may be provided, such as providing a four or three to one reduction in the number of signal paths. Data from 3 or 4 elements are combined onto a single output. Similar subarrays are formed for the entire array 12, such as associated with reducing 768 elements to 256 or 192 signal lines on the cable 26. Other relative amounts of reduction or subarray sizes may be used.

As another combination example, the active electronics 16 reduce the number of conductors 14 by combining signals using time division multiplexing or subarray mixing, or combinations thereof, such as implementing a mixer, multiplexer or other structures disclosed in U.S. Pat. No. ______ (application Ser. No. 10/741,827), (application Ser. No. 10/741,538), ______ (application Ser. No. 10/788,103), and/or ______ (application Ser. No. 10/834,779), the disclosures of which are incorporated herein by reference. The electronics by the arrays in the above patents are implemented as the active electronics 16. The electronics in the connector are still in the connector or in the handle 24. The active electronics 16 may additionally or alternatively implement multiplexers or other structures for configurable subarray groupings, such as grouping elements as a function of steering direction. By combining signals from a plurality of channels onto a fewer number of outputs, the channel count for output from the probe 10 and input to an imaging system is reduced. For example, the number of channels is reduced from at least 600 elements onto at most 300 outputs.

The transesophageal ultrasound probe 10 described above or a different transesophageal ultrasound probe provides a method for ultrasound imaging from within a patient. An electrical impedance of a plurality of signal conductors is substantially matched with a respective plurality of elements of a multi-dimensional transducer array by using multilayers of transducer material for each of the elements, such as two or more layers. Substantial matching is provided by increasing the capacitance as compared to a fewer number of layers of transducer material. An inexact match may be provided, such as the multilayer element having a lesser capacitance than the signal conductors or cables for connecting the element to active electronics. By establishing a capacitance of each of the elements with a plurality of layers, such as three or more layers of transducer material, a closer electrical impedance match between the elements and the signal conductors or cables is provided.

The more closely matched electrical impedance allows positioning of active electronics away from the transducer array. A smaller transesophageal probe than otherwise would be required is provided. The active electronics are positioned in a portion of the probe maintained outside of the patient, such as the handle. The active electronics electrically connect with the elements using signal conductors. Beamformer electronics, preamplifiers, multiplexers, mixers, combinations thereof or other active electronics are provided in the handle for operating the multi-dimensional transducer array. The active electronics in the multi-dimensional transducer array allow implementation of real time imaging, such as imaging using three-dimensional representations or a scan of a three-dimensional volume. The active electronics contribute to or control the scan within the three-dimensional volume using either receive, transmit or both receive and transmit beamformation or subarray formation. The imaging system completes any beamformation and generates three-dimensional representations from the scans of the volume at a substantially same time as the scan is happening as perceived by the user.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. An ultrasound transducer for imaging within an esophagus, the transducer comprising:
a transesophageal probe housing; and
a multi-dimensional transducer array in or on the transesophageal probe housing, the multi-dimensional transducer array having a plurality of elements, at least a first element of the plurality having multiple layers of transducer material.

2. The transducer of claim 1 wherein the transesophageal probe housing comprises a distal end, a handle and an articulating section between the distal end and the handle, the multi-dimensional transducer array being at or adjacent to the distal end, and the articulating section being steerable.

3. The transducer of claim 2 wherein the transesophageal probe housing is less than 16 mm in diameter at a maximum diameter from the distal end to the handle.

4. The transducer of claim 1 wherein the transesophageal probe housing comprises a distal end, a handle and a middle section between the handle and the distal end, the multi-dimensional transducer array being at or adjacent to the distal end, and wherein the distal end and the middle section are free of active electronics.

5. The transducer of claim 4 wherein the middle section is at least 40 cm in length.

6. The transducer of claim 4 further comprising active electronics in the handle, the active electronics electrically connected with the multi-dimensional transducer array and operable to combine signals from the plurality of elements onto a fewer number of outputs.

7. The transducer of claim 6 wherein plurality of elements comprises at least 600 elements;
further comprising:
at least 600 signal conductors connected with the at least 600 elements, respectively, and the active electronics;
wherein the active electronics are operable to combine signals from the at least 600 elements onto at most 300 outputs.

8. The transducer of claim 4 further comprising active electronics in the handle, the active electronics electrically connected with the multi-dimensional transducer array and operable to transmit and receive beamform.

9. The transducer of claim 1 wherein the first element comprises at least three layers of piezoelectric material.

10. The transducer of claim 1 wherein each element of the plurality of elements comprises multiple layers of transducer material.

11. The transducer of claim 1 wherein the multi-dimensional transducer array comprises a fully sampled or a sparse 2D array.

12. An ultrasound transducer for imaging within an esophagus, the transducer comprising:
a transesophageal probe housing having a distal end, a handle and a middle section between the handle and the distal end, the distal end and the middle section are free of active electronics;
a multi-dimensional transducer array at or adjacent to the distal end, the multi-dimensional transducer array having a plurality of elements; and
active electronics in the handle, the active electronics electrically connected with the multi-dimensional transducer array.

13. The transducer of claim 12 wherein the active electronics comprise a mixer, a multiplexer or combinations thereof to combine signals from the plurality of elements onto a fewer number of outputs.

14. The transducer of claim 13 wherein plurality of elements comprises at least 600 elements;
further comprising:
at least 600 signal conductors connected with the at least 600 elements, respectively, and the active electronics, the at least 600 signal conductors passing through the middle section;
wherein the active electronics are operable to combine signals from the at least 600 elements onto at most 300 outputs.

15. The transducer of claim 12 wherein the middle section is at least 40 cm in length.

16. The transducer of claim 12 wherein the active electronics comprise transmit, receive or both transmit and receive components.

17. The transducer of claim 12 wherein the transesophageal probe housing comprises an articulating section between the distal end and the handle, the articulating section being steerable.

18. The transducer of claim 12 wherein the transesophageal probe housing is less than 16 mm in diameter at a maximum diameter from the distal end to the handle.

19. The transducer of claim 12 wherein the elements each comprise multiple layers of transducer material.

20. The transducer of claim 12 wherein the multi-dimensional transducer array comprises: of a fully sampled or a sparse 2D array.

21. A method for ultrasound imaging from within a patient, the method comprising:
substantially matching an electrical impedance of a plurality of signal conductors with a respective plurality of elements of a multi-dimensional transducer array in a transesophageal ultrasound probe by each of the plurality of elements having at least two layers of transducer material; and
positioning active electronics in a portion of the transesophageal ultrasound probe maintained outside the patient, the active electronics electrically connected with the plurality of elements by the plurality of signal conductors.

22. The method of claim 21 further comprising:
maintaining portions of the transesophageal ultrasound probe insertable within the patient free of any active electronics.

23. The method of claim 22 wherein maintaining comprises providing the plurality of signal conductors along at least 40 cm of the transesophageal ultrasound probe from the multi-dimensional transducer array to the active electronics in the portion maintained outside the patient.

24. The method of claim 21 wherein substantially matching comprises establishing a capacitance of each of the plurality of elements with at least three layers of transducer material.

25. The method of claim 21 wherein positioning comprises positioning beamformer electronics in a handle of the transesophageal ultrasound probe.

26. The method of claim 21 wherein positioning comprises positioning preamplifiers, multiplexers, mixers or combinations thereof in the portion.

27. The method of claim 21 further comprising:
imaging in real-time with the active electronics and the multi-dimensional transducer array.
